(19) United States
(12) Patent Application Publication
(10) Pub. No.: US 2026/0026862 A1
(43) Pub. Date: Jan. 29, 2026

(54) ELECTROSURGICAL INSTRUMENT AND METHOD OF MONITORING CLAMP POSITION TO ADJUST ENERGY APPLICATION

(71) Applicant: Cilag GmbH International, Zug (CH)

(72) Inventors: Steven M. Boronyak, Cincinnati, OH (US); David J. Maher, Philadelphia, PA (US); Narendran Narasimhan, Cincinnati, OH (US); Madison K. Vanosdoll, Cincinnati, OH (US)

(21) Appl. No.: 18/783,680

(22) Filed: Jul. 25, 2024

(51) Int. Cl. A61B 18/14; A61B 18/00; A61B 90/00

(52) U.S. Cl. CPC A61B 18/1445; A61B 2018/0063; A61B 2018/00642; A61B 2018/00702; A61B 2018/00875; A61B 2018/00886; A61B 2018/1455; A61B 2090/061

(57) ABSTRACT

A surgical instrument and method of adjusting a power of a radiofrequency (RF) energy applied to a tissue includes an end effector and a controller. The end effector has a first jaw with a first electrode and a second jaw with a second electrode such that the first and second electrodes are configured to apply a radiofrequency (RF) energy through the tissue. The controller is configured to determine a jaw gap contraction rate while applying the RF energy to the tissue. Additionally, the controller is configured to compare the jaw gap contraction rate to at least one predetermined contraction rate threshold and then adjust a power of the RF energy while applying the RF energy to the tissue in response to the jaw gap contraction rate reaching the predetermined contraction rate threshold.

### BACKGROUND

A variety of surgical instruments include a tissue cutting element and one or more elements that transmit radio frequency (RF) energy to tissue (e.g., to coagulate or seal the tissue). An example of such an electrosurgical instrument is the ENSEAL® Tissue Sealing Device by Ethicon Endo-Surgery, Inc., of Cincinnati, Ohio. During use of such surgical instruments in one example, tissue is prepared, sealed with a desirable tissue seal, and severed for treatment. Generating the desirable tissue seal includes closely monitoring the state of the tissue while applying RF energy to the tissue during a power phase such that the tissue seals with relatively low damage and a relatively high burst pressure, in turn, delivering improved patient outcomes.

Unfortunately, applying too much or too little RF energy to the tissue for too long or too short of time tends to negatively impact the quality of the tissue seal. Many measurements are thus taken of the tissue in real-time for feedback of the tissue's state in an attempt to adjust application of the RF energy and increase the likelihood of achieving the desirable seal. While these real-time measurements, such as tissue impedance, have provided greater control of RF energy for more effective sealing, such measurements still provide little time to adjust the RF energy. Therefore, earlier indicators of tissue state and anticipated tissue sealing would provide even more time to adjust application of RF energy to the tissue in real-time for increasing the likelihood of achieving a desirable tissue seal.

While a variety of surgical instruments have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a perspective view of an exemplary electrosurgical instrument;

FIG. 2 depicts a perspective view of an exemplary articulation assembly and end effector of the electrosurgical instrument of FIG. 1;

FIG. 3 depicts an exploded view of the articulation assembly and end effector of FIG. 2;

FIG. 4 depicts a perspective view of the end effector that of FIG. 2;

FIG. 5 depicts an exploded perspective view of the end effector of FIG. 2;

FIG. 6 depicts an illustrative impedance triangle;

FIG. 7 depicts a flowchart of an example of a method of treating a tissue with the electrosurgical instrument of FIG. 1 including preparing the tissue, sealing the tissue, and severing the tissue;

FIG. 8A depicts a tissue received between jaws of the end effector in an open configuration;

FIG. 8B depicts the tissue received within the end effector similar to FIG. 8A, but with the jaws in a closed configuration and a relatively larger angle between the jaws receiving the method of treatment of FIG. 7;

FIG. 8C depicts the tissue received within the end effector similar to FIG. 8B, but with the jaws in the closed configuration and a relatively smaller angle between the jaws receiving the method of treatment of FIG. 7;

FIG. 9 depicts a flowchart of a first example of a method of sealing tissue based at least in part on monitoring a jaw gap contraction rate as a leading indicator of a desirable tissue seal while sealing;

FIG. 10 depicts an example graph of sealing tissue according to the method of FIG. 9 showing jaw gap, impedance, and temperature during treatment;

FIG. 11 depicts another example graph of sealing tissue according to the method of FIG. 9 showing impedance, power, voltage, and jaw gap with the jaw gap contraction rate being the leading indicator of the desirable tissue seal;

FIG. 12 depicts a flowchart of a second example of a method of sealing tissue based at least in part on monitoring jaw gap contraction rate while sealing as an indicator for terminating power;

FIG. 13 depicts an example graph of sealing tissue according to the method of FIG. 12 showing jaw gap contraction rate during treatment as an indicator for terminating power;

FIG. 14 depicts a flowchart of a third example of a method of sealing tissue based at least in part on monitoring jaw gap contraction rate while sealing as an indicator for terminating power;

FIG. 15 depicts a flowchart of a fourth example of a method of sealing tissue based at least in part on monitoring jaw gap contraction rate while sealing as an indicator for adjusting power;

FIG. 16 depicts an example graph of sealing tissue with a desirable jaw gap contraction rate, such as achieved via the method of FIG. 15;

FIG. 17 depicts an example graph of sealing tissue with a less desirable and relatively high jaw gap contraction rate; and

FIG. 18 depicts an example graph of sealing tissue with a less desirable and relatively low jaw gap contraction rate.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a surgeon or other operator grasping a surgical instrument having a distal surgical end effector. The term “proximal” refers the position of an element closer to the surgeon or other operator and the term “distal” refers to the position of an element closer to the surgical end effector of the surgical instrument and further away from the surgeon or other operator.

### I. Example of Electrosurgical Instrument

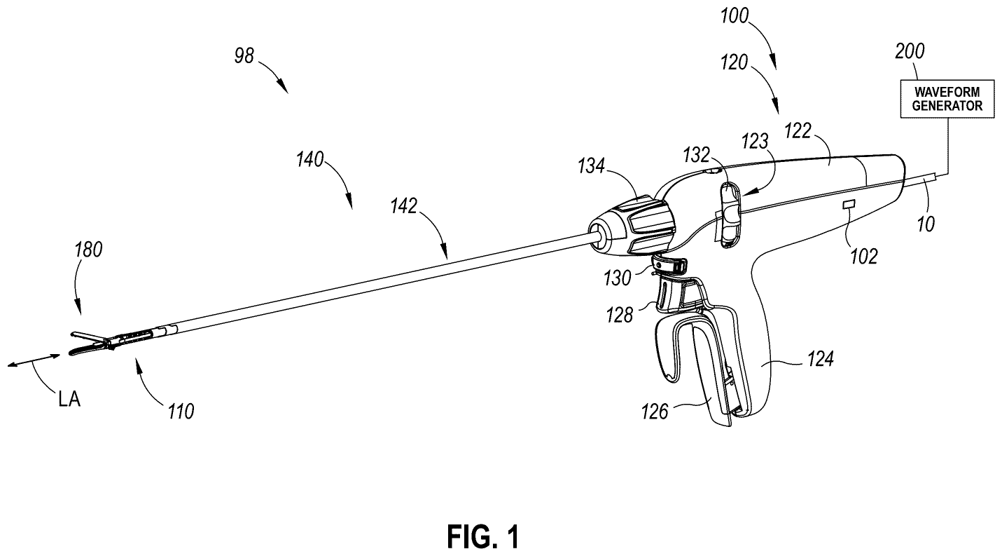

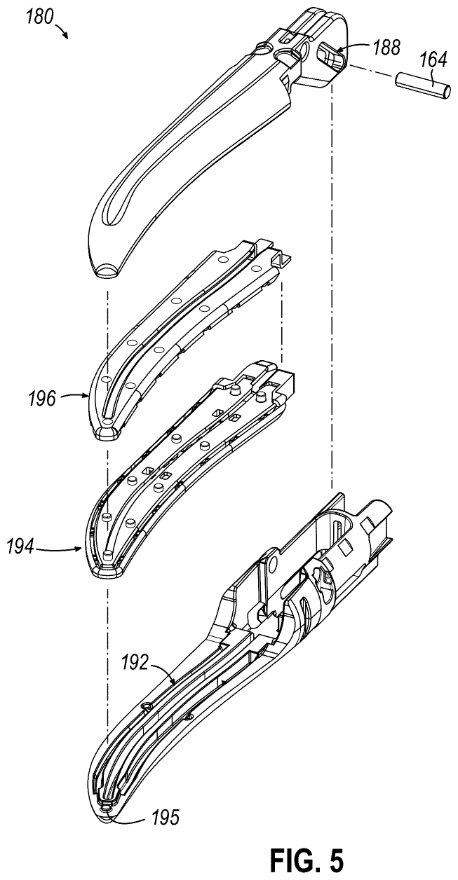

FIGS. 1-5 show a surgical system (98) including an exemplary electrosurgical instrument (100). As best seen in FIG. 1, electrosurgical instrument (100) includes a handle assembly (120), a shaft assembly (140), an articulation assembly (110), which may also be referred to as an articulation section (110), and an end effector (180). As will be described in greater detail below, end effector (180) of electrosurgical instrument (100) is operable to grasp, cut, and seal or weld tissue (e.g., a blood vessel, etc.). In this example, end effector (180) is configured to apply a non-therapeutic bipolar radio frequency (RF) energy in order to identify and/or verify that the correct tissue is present in the end effector such that a therapeutic RF energy can be applied to seal or weld tissue. However, it should be understood that electrosurgical instrument (100) may be configured to seal or weld tissue through any other suitable means that would be apparent to one skilled in the art in view of the teachings herein. For example, electrosurgical instrument (100) may be configured to seal or weld tissue via an ultrasonic blade, staples, etc. In the present example, electrosurgical instrument (100) is electrically coupled to a waveform generator (200) of surgical system (98), which is capable of delivering therapeutic and non-therapeutic energy, via power cable (10).

Waveform generator (200) may be configured to provide all or some of the electrical power requirements for use of electrosurgical instrument (100). Any suitable waveform generator (200) may be used as would be apparent to one skilled in the art in view of the teachings herein. By way of non-limiting example, the waveform generator (200) may be constructed in accordance with at least some of the teachings of U.S. Pat. No. 8,986,302, entitled “Surgical Generator for Ultrasonic and Electrosurgical Devices,” issued Mar. 24, 2015, the disclosure of which is incorporated by reference herein, in its entirety. While in the current example, electrosurgical instrument (100) is coupled to waveform generator (200) via power cable (10), electrosurgical instrument (100) may contain an internal power source or plurality of power sources, such as a battery and/or supercapacitors, to electrically power electrosurgical instrument (100). Of course, any suitable combination of power sources may be utilized to power electrosurgical instrument (100) as would be apparent to one skilled in the art in view of the teaching herein.

Handle assembly (120) is configured to be grasped by an operator with one hand, such that an operator may control and manipulate electrosurgical instrument (100) with a single hand. Although electrosurgical instrument (100) is primarily described herein as being used by a human user, it should be noted that alternative versions exist in which one or more robotic systems (e.g., a robotic arm) may be used to control and manipulate electrosurgical instrument (100). Shaft assembly (140) extends distally from handle assembly (120) and connects to articulation assembly (110). Articulation assembly (110) is also connected to a proximal end of end effector (180). As will be described in greater detail below, components of handle assembly (120) are configured to control end effector (180) such that an operator may grasp, cut, and seal or weld tissue. Articulation assembly (110) is configured to deflect end effector (180) from the longitudinal axis (LA) defined by shaft assembly (140).

Handle assembly (120) of the present example includes a control unit (102), which may also be referred to herein as a controller (102), housed within a body (122), a pistol grip (124), a jaw closure trigger (126), a knife trigger (128), an activation button (130), an articulation control (132), and a knob (134). As will be described in greater detail below, jaw closure trigger (126) may be pivoted toward and away from pistol grip (124) and/or body (122) to open and close jaws (182, 184) of end effector (180) to grasp tissue. Additionally, knife trigger (128) may be pivoted toward and away from pistol grip (124) and/or body (122) to actuate a knife member (176) within the confines of jaws (182, 184) to cut tissue captured between jaws (182, 184). Further, activation button (130) may be pressed to apply radio frequency (RF) energy to tissue via electrodes (194, 196) of jaws (182, 184), respectively. In some versions, electrodes (194, 196) of jaws (182, 184) are in a bifurcation configuration where electrodes (194, 196) move relative to a central axis and nearly equal and opposite to one another.

Body (122) of handle assembly (120) defines an opening (123) through which a portion of articulation control (132) protrudes. Articulation control (132) is rotatably disposed within body (122) such that an operator may rotate the portion of articulation control (132) protruding from opening (123) to rotate the portion of articulation control (132) located within body (122). Rotation of articulation control (132) relative to body (122) will bend articulation assembly (110) in order to drive deflection of end effector (180) from the longitudinal axis (LA) defined by shaft assembly (140). Articulation control (132) and articulation assembly (110) may include any suitable features to drive deflection of end effector (180) from the longitudinal axis (LA) defined by shaft assembly (140) as would be apparent to one skilled in the art in view of the teachings herein.

Knob (134) is rotatably disposed on the distal end of body (122) and is configured to rotate end effector (180), articulation assembly (110), and shaft assembly (140) about the longitudinal axis (LA) of shaft assembly (140) relative to handle assembly (120). While in the current example, end effector (180), articulation assembly (110), and shaft assembly (140) are rotated by knob (134), knob (134) may be configured to rotate end effector (180) and articulation assembly (110) relative to selected portions of shaft assembly (140). Knob (134) may include any suitable features to rotate end effector (180), articulation assembly (110), and shaft assembly (140) as would be apparent to one skilled in the art in view of the teachings herein.

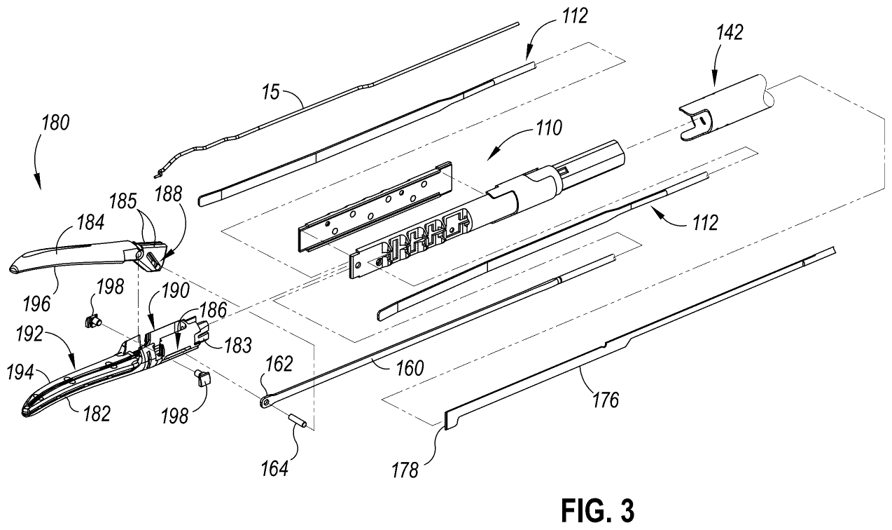

Shaft assembly (140) includes distal portion (142) extending distally from handle assembly (120) and a proximal portion housed within the confines of body (122) of handle assembly (120). Referring to FIG. 3, shaft assembly (140) houses a jaw closure connector (160) that couples jaw closure trigger (126) with end effector (180). Additionally, shaft assembly (140) houses a portion of knife member (176) extending between distal a distal cutting edge (178) of knife member (176) and knife trigger (128). Shaft assembly (140) also houses actuating members (112) that couple articulation assembly (110) with articulation control (132); as well as an electrical coupling (15) that operatively couples electrodes (194, 196) with activation button (130). As will be described in greater detail below, jaw closure connector (160) is configured to translate relative to shaft assembly (140) to open and close jaws (182, 184) of end effector (180); while knife member (176) is coupled to knife trigger (128) of handle assembly (120) to translate distal cutting edge (178) within the confines of end effector (180); and activation button (130) is configured to activate electrodes (194, 196).

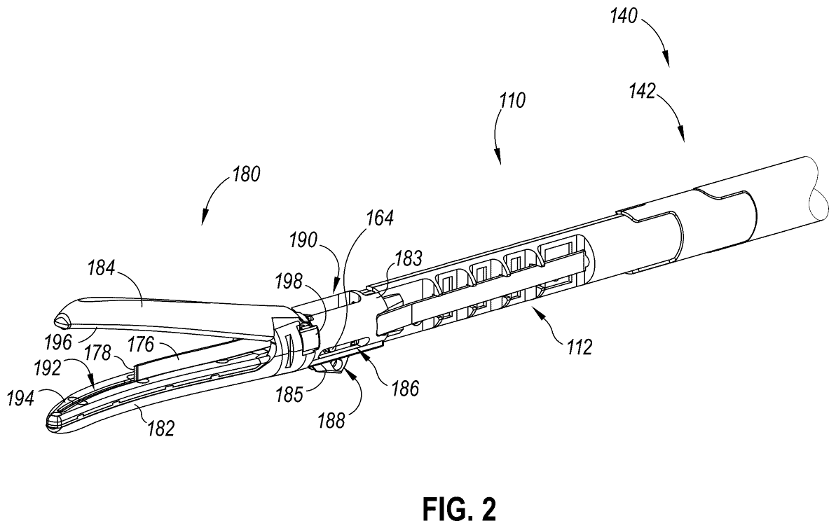

As best seen in FIGS. 2-5, end effector (180) includes lower jaw (182) pivotally coupled with upper jaw (184) via pivot couplings (198). Lower jaw (182) includes a proximal body (183) defining a slot (186), while upper jaw (184) includes proximal arms (185) defining a slot (188). Lower jaw (182) also defines a central channel (190) that is configured to receive proximal arms (185) of upper jaw (184), portions of knife member (176), jaw closure connector (160), and pin (164). Slots (186, 188) each slidably receive pin (164), which is attached to a distal coupling portion (162) of jaw closure connector (160). Additionally, lower jaw (182) includes a force sensor (195) located at a distal tip of lower jaw (182), though force sensor (195) may alternatively be positioned at any other suitable location. Force sensor (195) may be in communication with control unit (102). Force sensor (195) may be configured to measure the closure force generated by pivoting jaws (182, 184) into a closed configuration in accordance with the description herein. Additionally, force sensor (195) may communicate this data to control unit (102). Any suitable components may be used for force sensor (195) as would be apparent to one skilled in art in view of the teachings herein. For example, force sensor (195) may take the form of a strain gauge. In some variations, end effector (180) includes more than one force sensor.

While in the current example, a force sensor (195) is incorporated into electrosurgical instrument (100) and is in communication with control unit (102), any other suitable sensors or feedback mechanisms may be additionally or alternatively incorporated into electrosurgical instrument (100) while in communication with control unit (102) as would be apparent to one skilled in the art in view of the teachings herein. For instance, an articulation sensor or feedback mechanism may be incorporated into electrosurgical instrument (100), where the articulation sensor communicates signals to control unit (102) indicative of the degree end effector 180 is deflected from the longitudinal axis (LA) by articulation control (132) and articulation assembly (110).

As will be described in greater detail below, jaw closure connector (160) is operable to translate within central channel (190) of lower jaw (182). Translation of jaw closure connector (160) drives pin (164). As will also be described in greater detail below, with pin (164) being located within both slots (186, 188), and with slots (186, 188) being angled relative to each other, pin (164) cams against proximal arms (185) to pivot upper jaw (184) toward and away from lower jaw (182) about pivot couplings (198). Therefore, upper jaw (184) is configured to pivot toward and away from lower jaw (182) about pivot couplings (198) to grasp tissue.

The term “pivot” does not necessarily require rotation about a fixed axis and may include rotation about an axis that moves relative to end effector (180). Therefore, the axis at which upper jaw (184) pivots about lower jaw (182) may translate relative to both upper jaw (184) and lower jaw (182). Any suitable translation of the pivot axis may be used as would be apparent to one skilled in the art in view of the teachings herein.

Lower jaw (182) and upper jaw (184) also define a knife pathway (192). Knife pathway (192) is configured to slidably receive knife member (176), such that knife member (176) may be retracted, and advanced, to cut tissue captured between jaws (182, 184).

Lower jaw (182) and upper jaw (184) each comprise respective electrodes (194, 196). The power source may provide RF energy to electrodes (194, 196) via electrical coupling (15) that extends through handle assembly (120), shaft assembly (140), articulation assembly (110), and electrically couples with one or both of electrodes (194, 196). Electrical coupling (15) may selectively activate electrodes (194, 196) in response to an operator pressing activation button (130). In some instances, control unit (102) may couple electrical coupling (15) with activation button (130), such that control unit (102) activates electrodes (194, 196) in response to operator pressing activation button (130). Control unit (102) may have any suitable components in order to perform suitable functions as would be apparent to one skilled in the art in view of the teachings herein. For instance, control unit (102) may have a processor, memory unit, suitable circuitry, etc. Examples of features and functionalities that may be incorporated into control unit (102) will be described in greater detail below.

As described above, jaw closure trigger (126) may be pivoted toward and away from pistol grip (124) and/or body (122) to open and close jaws (182, 184) of end effector (180) to grasp tissue. In particular, as will be described in greater detail below, pivoting jaw closure trigger (126) toward pistol grip (124) may proximally actuate jaw closure connector (160) and pin (164), which in turn cams against slots (188) of proximal arms (185) of upper jaw (184), thereby rotating upper jaw (184) about pivot couplings (198) toward lower jaw (182) such that jaws (182, 184) achieve a closed configuration.

In some versions, knife trigger (128) may be pivoted toward and away from body (122) and/or pistol grip (124) to actuate knife member (176) within knife pathway (192) of jaws (182, 184) to cut tissue captured between jaws (182, 184). In particular, handle assembly (120) further includes a knife coupling body that is slidably coupled along proximal portion of shaft assembly (140). Knife coupling body is coupled with knife member (176) such that translation of knife coupling body relative to proximal portion of shaft assembly (140) translates knife member (176) relative to shaft assembly (140).

In another version, knife coupling body may be coupled to a knife actuation assembly such that as knife trigger (128) pivots toward body (122) and/or pistol grip (124), knife actuation assembly drives knife coupling body distally, thereby driving knife member (176) distally within knife pathway (192). Because knife coupling body is coupled to knife member (176), knife member (176) translates distally within shaft assembly (140), articulation assembly (110), and within knife pathway (192) of end effector (180). Knife member (176) includes distal cutting edge (178) that is configured to sever tissue captured between jaws (182, 184). Therefore, pivoting knife trigger (128) causes knife member (176) to actuate within knife pathway (192) of end effector (180) to sever tissue captured between jaws (182, 184).

With distal cutting edge (178) of knife member (176) actuated to the advanced position, an operator may press activation button (130) to selectively activate electrodes (194, 196) of jaws (182, 184) to seal or weld severed tissue captured between jaws (182, 184). It should be understood that the operator may also press activation button (130) to selectively activate electrodes (194, 196) of jaws (182, 184) at any suitable time during exemplary use. Therefore, the operator may also press activation button (130) while knife member (176) is retracted. Next, the operator may release jaw closure trigger (126) such that jaws (182, 184) pivot into the opened configuration, releasing tissue.

### II. Sensing Tissue Impedance for Determinations of Tissue State

Electrosurgical instrument (100) discussed above is configured to clamp tissue using end effector (180). Once securely clamped, electrodes (194, 196) in end effector (180) apply a non-therapeutic (i.e., low voltage) waveform to the tissue; and sensor devices measure the returning waveform to calculate and measure the impedance of the tissue. In one example, one or more electrodes (194, 196) are operatively connected to such sensor devices such that electrodes (194, 196) may be referred to as sensors in this respect. More specifically, electrosurgical instrument (100), via one or more sub-circuits, will provide non-therapeutic energy to the extracellular and intracellular fluid present within a given (e.g., clamped) region of tissue to determine a phase and a magnitude of the impedance of the tissue within jaws (182, 184). A processor may then relay information associated with a state of the tissue, such as, for example, tissue type, tissue phase, tissue margin, and the like. Using this associated information, a system inclusive of such electrosurgical instrument (100) cannot only verify that the proper tissue is clamped between jaws (182, 184), but can also determine if any non-tissue material is present between jaws (182, 184), and/or if a proper seal has been created after applying the therapeutic RF energy.

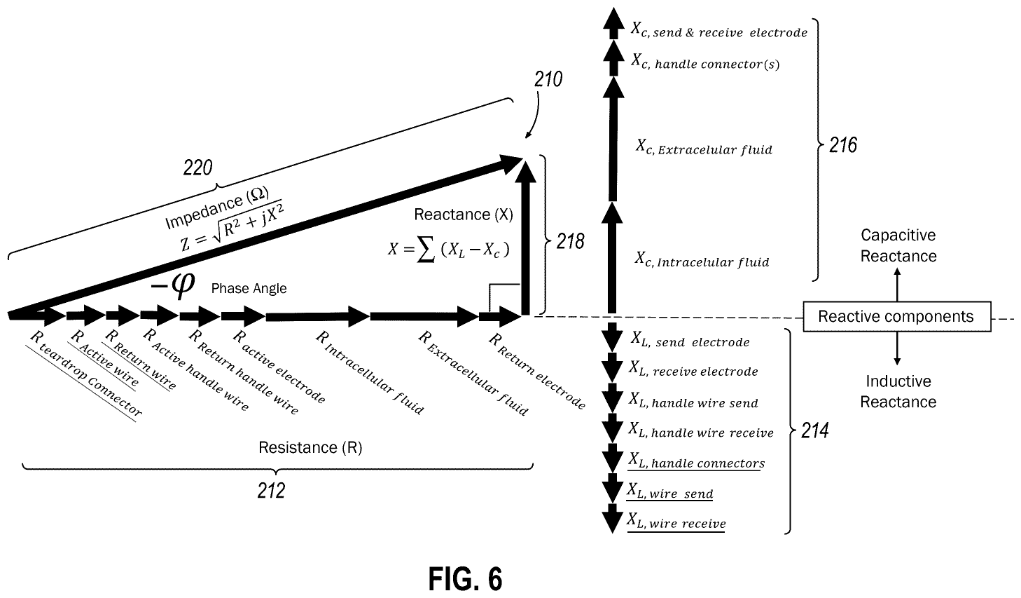

FIG. 6 shows an illustrative impedance triangle (210). As would be understood by one skilled in the art, human tissues may tend to be capacitive in nature, while wires, tool, staples, implants, etc. may tend to be inductive in nature. Thus, as can be seen by the illustrative impedance triangle (210), the “resistance” of each object in the circuit is measured (212) using the waveform and sensor electrodes (194, 196). Such system can also determine the “capacitive reactance” of each object in the circuit and the inductive reactance of each object in the circuit. For example, the send and receive electrodes (194, 196), the send and receive handle wires, the handle connector and the send and receive wires (e.g., included in power cable 10 (see FIG. 1)) all have inductive reactance (214). Additionally, the send and receive electrodes (194, 196), the handle connector, the extracellular fluid, and the intracellular fluid all have capacitive reactance (216). The “reactance” (218) can then be calculated by determining the difference between the capacitive reactance and the inductive reactance using:

$$X = \sum ( {X}_{L} - {X}_{C} ) . \qquad Equation 1$$

As shown in FIG. 6, the “impedance” 1406 can then be determined using:

$$Z = \sqrt{{R}^{2} + {jX}^{2}} \qquad Equation 2$$

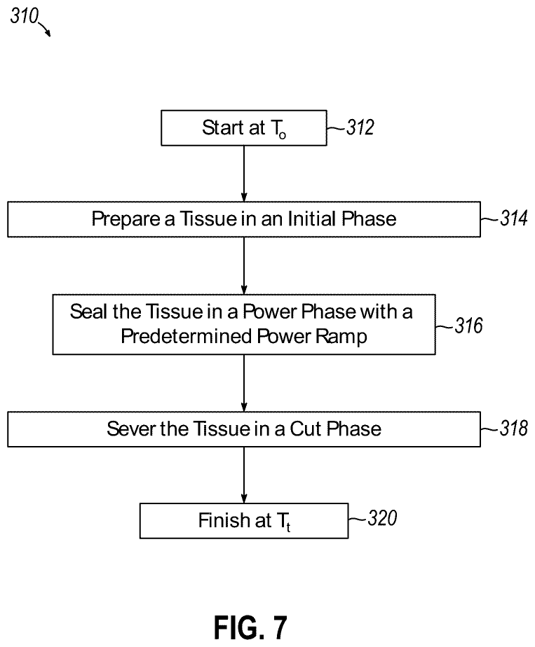

FIG. 7 shows a set of illustrative example waveforms. As would be understood by one skilled in the art, if a circuit only contains resistive items, the current and voltage will remain in phase such as shown in a first graph (230) and a first phasor diagram (231). Alternatively, if the circuit has capacitive objects, or more capacitive than inductive, the voltage wave will lead the current wave such as shown in a second graph (232) and a second phasor diagram (233). Finally, if the circuit has inductive objects, or more inductive objects than capacitive objects, the voltage will lag behind the current, such as shown in a third graph (234) and third phasor diagram (235).

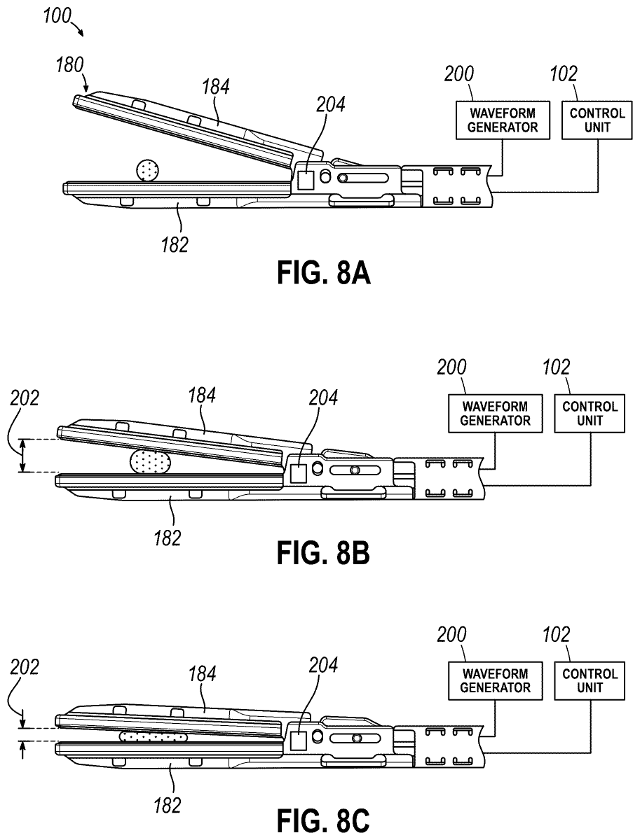

As discussed herein, the system may pass a non-therapeutic waveform through a portion of patient tissue to help identify the type of tissue as well as any foreign objects. Thus, in some versions, the system may pass waveforms of varying frequency (e.g., in series and/or parallel) to improve the accuracy of the determination. Accordingly, in some versions, and as shown in FIG. 8, multiple waveforms of various frequencies may be added or summed together (240) to create a muti-sine waveform (241). By way of non-limiting example, a 10 kHz sine wave (242) may be combined with a 100 kHz sine wave (243), a 330 kHz sine wave (244) and a 1 MHz sine wave (245) may be combined to create multi-sine wave (241).

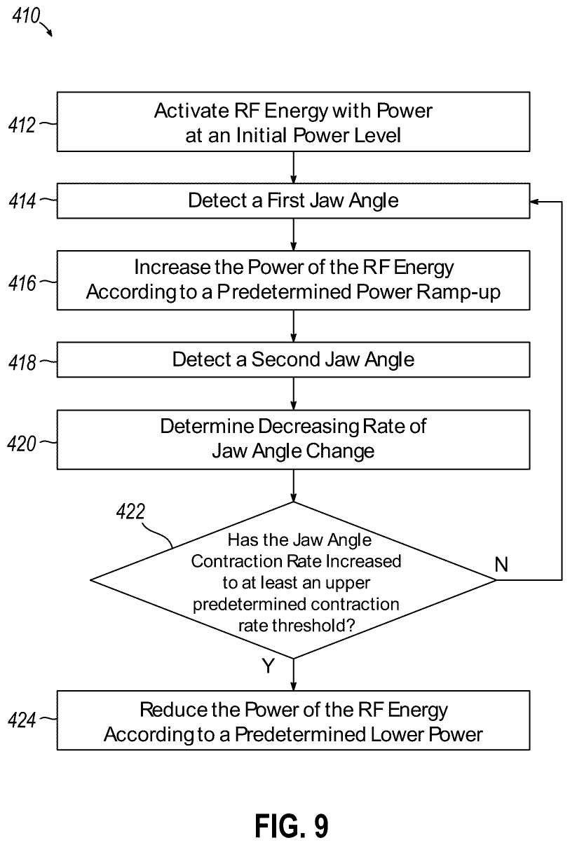

Referring now to FIG. 9, multi-sine waveform (241) may be sampled or windowed. In some versions, such as those that require the use of Fast Fourier Transforms (FFT), the windowing or sampling may be as small as a single period for the lower frequency waveform. As shown in a fourth graph (250), the voltage of multi-sine waveform (241) is leading the current and thus in the present version indicates a capacitive circuit (e.g., likely tissue). In an alternative version, the system may apply a series of burst waveforms having different frequencies.

Referring now to FIG. 10, a burst waveform, including a brief delay between frequencies, is shown in fifth graph (260). In some versions, and as shown, the system may output a burst waveform that is a sine wave, while in other versions, the wave may be a square, triangle, ramp, pulse, pseudorandom binary sequence (PRBS), or arbitrary waveform. In some versions, the pause between waveforms can be evaluated in order to determine a “rebounding” time. The rebounding time may be used to help identify tissue types by evaluating how long certain tissues take to allow the waveform and any residual energy to dissipate from the tissue.

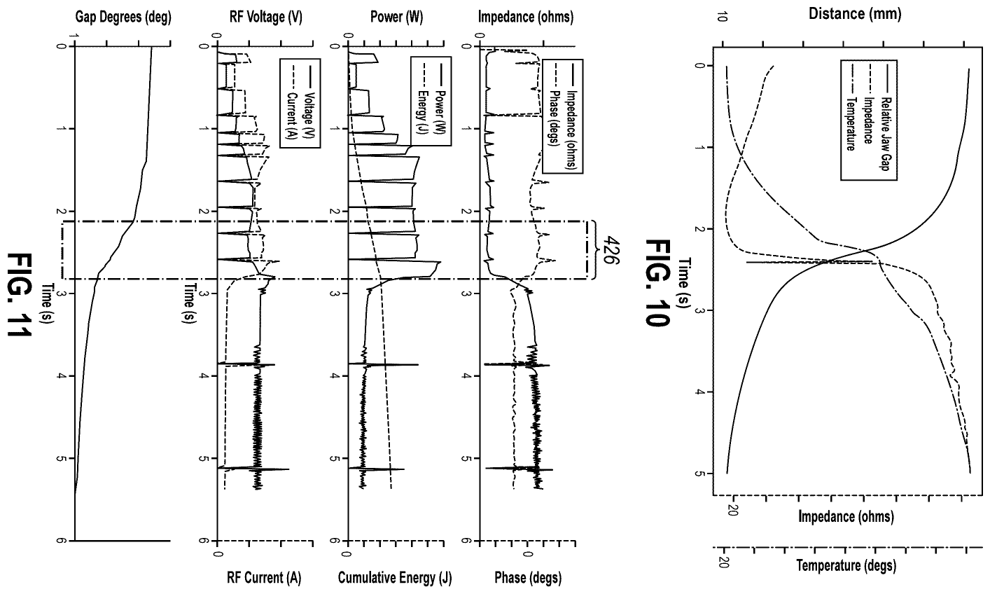

FIG. 11 shows various alternative burst versions. Specifically, in one version, amplitude modulation (AM) (262) may be used; while in another version, frequency modulation (FM) (264). Other versions may use phase modulation (PM) (266) and/or frequency-shift keying (FSK) modulation (268). Due to the fact that all of the modulation options shown in FIG. 11 involve a shift of some type, they may all be evaluated in a similar manner.

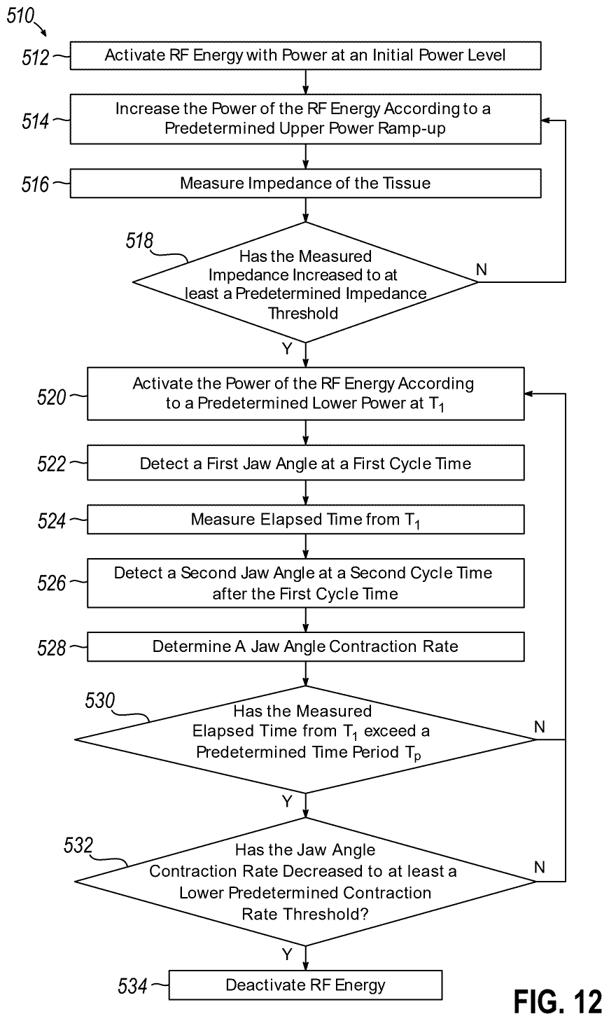

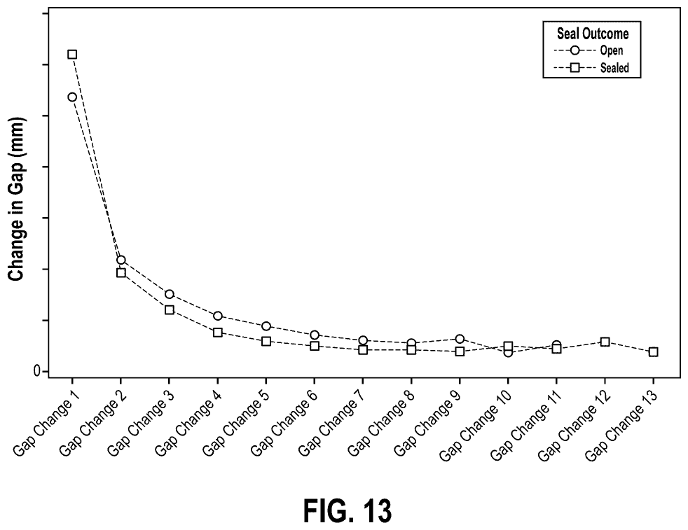

In a further version, a “chirp” function can be used, such as shown in FIG. 12. As would be understood by one skilled in the art, a chirp wave can be an “up-chirp” (i.e., the frequency increases) or a “down-chirp” (i.e., the frequency decreases). Thus, stated differently, a chirp function is essentially an advanced form of FM (264). The chirp function shown in a sixth graph (270) shows a chirp waveform with increasing frequency (e.g., 10 kHz, 13.2 kHz, 19.3 kHz, 26.8 kHz, and 1 Mhz). FIG. 13 shows a seventh graph (272) depicting a chirp function with the same frequencies as shown in FIG. 12, but with a decreasing amplitude. Additional features associated with electrical circuits and measurements of tissue are described in U.S. patent application Ser. No. 17/854,306, entitled “Electrosurgical Instrument for Applying Non-Therapeutic RF Signals,” filed Jun. 30, 2022, and published as U.S. Pat. Pub. No. 2024/0000499 on Jan. 4, 2024, the disclosure of which is incorporated by reference herein, in its entirety.

### III. Electrosurgical Instrument and Method of Tissue Treatment

Electrosurgical instrument (100) performs one example of a method (310) of treating tissue of a patient as shown in FIGS. 7-8C. Generally, jaws (182, 184) receive tissue in the open configuration. The operator then directs jaw (182, 184) toward the closed configuration thereby compressing the tissue therebetween at an initial start time, To, in a step (312). Once clamped, at the operator's continued direction, control unit (102) prepares the tissue in an initial phase of treatment in a step (314). One example of such tissue preparation includes applying subtherapeutic RF energy to the tissue via electrodes (194, 196) to determine one or more classifications of the tissue (e.g., type of tissue, size of tissue, etc.) to inform later steps of treatment that may be unique to particular tissues. After preparation, control unit (102) directs waveform generator (200) to apply therapeutic RF energy to the tissue via electrodes (194, 196) to seal the tissue in a power phase in a step (316), such as discussed below in greater detail. With the tissue desirably sealed, knife member (176) is fired as directed by the operator and/or control unit (102) thereby severing the tissue in a cut phase in a step (318) and finishing at least this portion of the treatment method (310) in a step (320).

In an attempt to more accurately determine when tissue is satisfactorily sealed during the power phase, such as with a desired, predetermined tissue seal, additional measurements have been explored. One such approach uses a physical measurement of the jaw gap (e.g., jaw aperture, jaw angle), of electrosurgical instrument (100), during activation of the electrodes (194, 196) in the power phase, to control the activation. Following this approach, the lower the jaw angle measurement, the more likely that the tissue seal is complete. However, tissues comprise varying amounts of tissue constituents (e.g., proteins) that do not vaporize or melt away, or change states at different rates, causing the jaw angle to change in an erratic way. As a result, any instantaneous jaw angle measurement may not provide sufficient information to infer seal completeness to the predetermined tissue seal. As used herein, references to seals being “desired,” “desirable,” “complete,” or “predetermined” refer to sealing tissue with relatively low damage and a relatively high burst pressure as desired, such as for a particular tissue type. While such desired, predetermined tissue seal for complete sealing may be targeted for a given tissue, it will be appreciated that a desirable, predetermined tissue seal may nonetheless vary depending on a particular tissue type or use such that the invention is not intended to be unnecessarily limited to a specific predetermined tissue seal.

In one example, with respect to FIGS. 8B and 8C, the tissue is compressed at initiation of the power phase, but with a relatively larger cross-sectional area before application of the therapeutic RF energy such that the jaws (182, 184) have a relatively large jaw gap (202) therebetween in the closed configuration. While the therapeutic RF energy is being applied, denaturing of the tissue causes this cross-sectional area of tissue to decrease and, in turn, jaw gap (202) between jaws (182, 184) decreases while continuing to compress the tissue until completion of the power phase in step (318). In order to monitor this jaw gap (202) during sealing, a jaw sensor (240) is incorporated into end effector (180) and operatively connected between at least one of jaws (182, 184) and control unit (102). Jaw sensor (240) is thus configured to monitor the position of one of jaws (182, 184) relative to the other of jaws (184, 182) such that control unit (102) measures jaw gap (202). More particularly, in one example, jaw gap (202) is measured as an angle between jaws (184, 182), but any alternative measurement between jaws (184, 182) may be similarly monitored. In any case, measurements of jaw gap (202) may be useful in one or more methods of sealing tissue for determining whether or not a desirable seal has formed in tissue during the power phase in step (318).

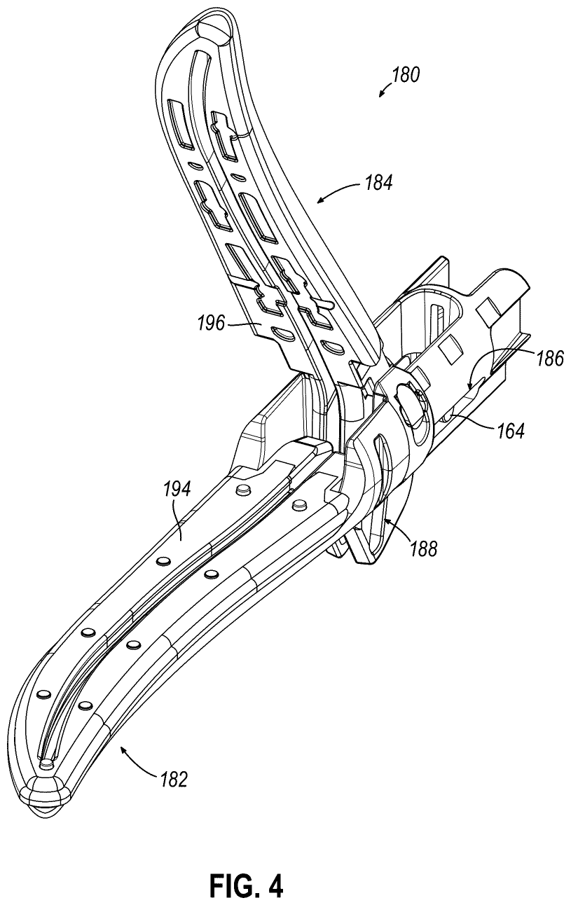

In contrast to simply taking instantaneous measurements of jaw gap (202), a rate of change of jaw gap (202) over time during the power phase provides even greater understanding of the state of the tissue in real-time for achieving desirable, predetermined seals in tissue as discussed below in any one or more of methods (410, 510, 610, 710). This rate of change of jaw gap (202) is referred to herein as a “jaw gap contraction rate” and, more particularly, as a “jaw angle contraction rate.” Any one or more steps of methods (410, 510, 610, 710) may be incorporated into the overall treatment of tissue, such as method (310) discussed above, such that the invention is not intended to be unnecessarily limited to a particular example of methods (410, 510, 610, 710) provided herein. Furthermore, like numbers discussed below indicate like features described above such that any reference to features of electrosurgical instrument (100) are in reference to FIGS. 4 and 8A-8C.

### A. Jaw Angle Contraction Rate as Leading Indicator of Power Adjustment Prior to Desirable Tissue Seal

With respect to FIGS. 9-11, an example of a method (410) of adjusting RF energy during the power phase of treatment method (310) includes activating the RF energy with power at an initial power level in a step (412) and detecting a first jaw angle in a step (414) at a first time with jaw sensor (204) via control unit (102). From the onset of this power phase with the initial power level, control unit (102) then increases the power of the RF energy being applied to the tissue at electrodes (194, 196) according to a predetermined power ramp-up. While RF energy is being applied to the tissue, jaw angle continues to contract as described above, therefore, a second jaw angle is detected in a step (418) at a second time. After the second jaw angle is detected and received by control unit (102), control unit (102) determines a jaw angle contraction rate that occurred from the times that each of the first and second jaws angles were detected in a step (420).

A memory unit of control unit (102) has an upper predetermined contraction rate threshold stored thereon. This upper predetermined contraction rate threshold represents an early indication of tissue approaching the desired seal parameters, such as a desired burst pressure. Therefore, control unit (102) compares the determined jaw angle contraction rate of step (420) to the upper predetermined contraction rate threshold and determines whether or not the determined jaw angle contraction rate has increased to at least the upper predetermined contraction rate threshold in a step (422). In the event that the jaw angle contraction rate has not yet reached the upper predetermined contraction rate threshold, control unit (102) again performs step (414), step (416), step (418), step (420), and step (422). However, in the event that the jaw angle contraction rate has reached the upper predetermined contraction rate threshold, then control unit (102) reduces the power of the RF energy according to a predetermined lower power in a step (424) in anticipation of achieving the desirable tissue seal. Earlier anticipation of achieving the desirable tissue seal allows for more effectively timing this power reduction to reduce the likelihood of overwhelming the tissue with too much RF energy too quickly and missing the desirable tissue seal.

FIG. 11 depicts this phenomenon of jaw angle contraction rate being a leading indicator as compared to measured impedance, which may otherwise indicate the desire tissue seal as a rise in impedance approaches a threshold impedance value. Notably, in the present example, measured impedance increases toward this threshold impedance value approximately 2.7 seconds after activating the RF energy, whereas the jaw angle contraction rate increases to its upper predetermined contraction rate threshold approximately 2.1 seconds after activating the RF energy. Therefore, jaw angle contraction rate provides approximately 0.6 seconds of additional time (426) to reduce the power of RF energy and improve the likelihood of achieving the desirable tissue seal. Of course, alternative times and outcomes may vary in alternative examples, such as with alternative tissues, such that the invention is not intended to be unnecessarily limited to the particular results shown in FIGS. 10 and 11.

While the above referenced examples of various thresholds stored on memory unit of control unit (102) may each be respectively accessed and applied to a variety of tissues for treatment, in another example, different predetermined thresholds may also be stored on memory unit of control unit (102) for selection during operation based on feedback from the subtherapeutic RF energy applied while preparing the tissue in the initial phase of step (314) (see FIG. 7). For example, control unit (102) may select one predetermined threshold over another predetermined threshold based on a characteristic of the tissue, such as tissue size and/or tissue type. The invention is therefore not intended to be unnecessarily limited to any one threshold for application to tissue.

### B. Jaw Angle Contraction Rate as an Indicator for Terminating a Power Phase

With respect to FIGS. 12-13, an example of a method (510) of adjusting RF energy during the power phase of treatment method (310) is shown that includes activating the RF energy with power at an initial power level in a step (512) and increasing the power of the RF energy according to a predetermined upper power-ramp up in a step (514). The control unit (102) then measures impedance of the tissue in a step (516). The memory unit of control unit (102) has a predetermined impedance threshold stored thereon. Thus, following measurement of the impedance of the tissue in step (516), control unit (102) compares the measured impedance to the predetermined impedance threshold. In the event that the measured impedance is less than the predetermined impedance threshold, then control unit (102) again performs step (514), step (516), and step (518). However, in the event that the measured impedance is at least the predetermined impedance threshold, then control unit decreases the power activation according to a predetermined lower power at a first time, T₁, in a step (520) for achieving the desirable tissue seal.

Control unit (102) directs continued powering of the RF energy until at least a minimum amount of time has passed and jaw angle contraction rate has decreased to at least a lower predetermined contraction rate threshold, which is stored on the memory unit of control unit (102). More particularly, following step (520) discussed above, control unit (102) detects a first jaw angle in a step (522) at a first cycle time with jaw sensor (204). The control unit (102) then measures an elapsed time from the first time, T₁, in a step (524). While RF energy is being applied to the tissue, jaw angle continues to contract as described above, therefore, a second jaw angle is detected in a step (528) at a second cycle time after the first cycle time. After the second jaw angle is detected and received by control unit (102), control unit (102) determines a jaw angle contraction rate that occurred from the times that each of the first and second jaws angles were detected.

In the present example, control unit (102) compares the measured elapsed time from the first time, T₁, to a predetermined time period, Tₚ, which is stored on the memory unit of control unit (102) in a step (530). In the event that the measured elapsed time is less than the predetermined time period, Tₚ, in step (530), then control unit (102) again performs step (520), step (522), step (524), step (526), step (528), and step (530). However, in the event that the measured elapsed time is at least the predetermined time period, Tₚ, in step (530), then control unit (102) compares the jaw angle contraction rate to a lower predetermined contraction rate threshold in a step (532). In the event that the jaw angle contraction rate has not yet decreased to the lower predetermined contraction rate threshold, control unit (102) again performs step (520), step (522), step (524), step (526), step (528), step (530), and step (532). However, in the event that the jaw angle contraction rate has decreased to at least the lower predetermined contraction rate threshold, then control unit (102) deactivates the RF energy in a step (534), thereby terminating the power phase with the tissue having the desirable tissue seal.

FIG. 13 depicts achieving the desirable tissue seal based on applying RF energy until at least a minimum amount of time has passed and jaw angle contraction rate has decreased to at least the lower predetermined contraction rate threshold. Of course, alternative gap changes and outcomes may vary in alternative examples, such as with alternative tissues, such that the invention is not intended to be unnecessarily limited to the particular results shown in FIG. 13.

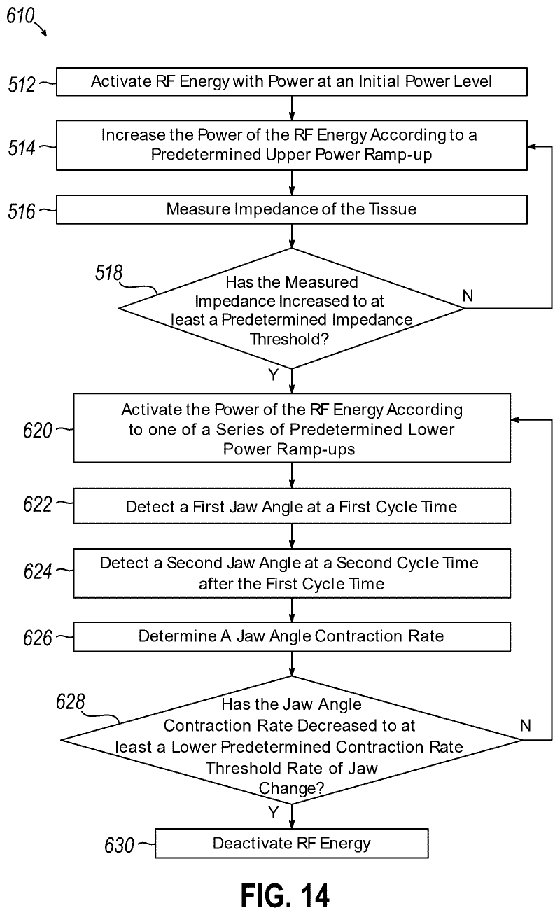

FIG. 14 depicts yet another example of a method (610) of adjusting RF energy during the power phase of treatment method (310) that includes activating the RF energy with power at an initial power level in step (512) and increasing the power of the RF energy according to a predetermined upper power-ramp up in step (514). In this respect method (610), like method (510) discussed above, follows step (512), step (514), step (516), and step (518). However, in the event that the measured impedance is at least the predetermined impedance threshold, then control unit decreases the power activation according to one of a series of predetermined lower power ramp-ups in a step (620) for achieving the desirable tissue seal.

Control unit (102) directs continued powering of the RF energy with one of a series of predetermined lower power ramp-ups until jaw angle contraction rate has decreased to at least a lower predetermined contraction rate threshold, which is stored on the memory unit of control unit (102). More particularly, following step (518) discussed above, control unit (102) detects a first jaw angle in a step (622) at a first cycle time with jaw sensor (204). While RF energy is being applied to the tissue, jaw angle continues to contract as described above, therefore, a second jaw angle is detected in a step (624) at a second cycle time after the first cycle time. After the second jaw angle is detected and received by control unit (102), control unit (102) determines a jaw angle contraction rate that occurred from the times that each of the first and second jaw angles were detected in a step (626).

In the present example, control unit (102) compares the jaw angle contraction rate to a lower predetermined contraction rate threshold in a step (628). In the event that the jaw angle contraction rate has not yet decreased to the lower predetermined contraction rate threshold, control unit (102) again performs step (620), which directs RF energy to another one of a series of predetermined lower power ramp-ups, step (622), step (624), and step (626). However, in the event that the jaw angle contraction rate has decreased to at least the lower predetermined contraction rate threshold, then control unit (102) deactivates the RF energy in a step (630), thereby terminating the power phase with the tissue having the desirable tissue seal.

While the above referenced examples of various thresholds stored on memory unit of control unit (102) may each be respectively accessed and applied to a variety of tissues for treatment, in another example, different predetermined thresholds may also be stored on memory unit of control unit (102) for selection during operation based on feedback from the subtherapeutic RF energy applied while preparing the tissue in the initial phase of step (314) (see FIG. 7). For example, control unit (102) may select one predetermined threshold over another predetermined threshold based on a characteristic of the tissue, such as tissue size and/or tissue type. The invention is therefore not intended to be unnecessarily limited to any one threshold for application to tissue.

### C. Jaw Angle Contraction Rate as an Indicator for Selecting a Predetermined Power Ramp-Up

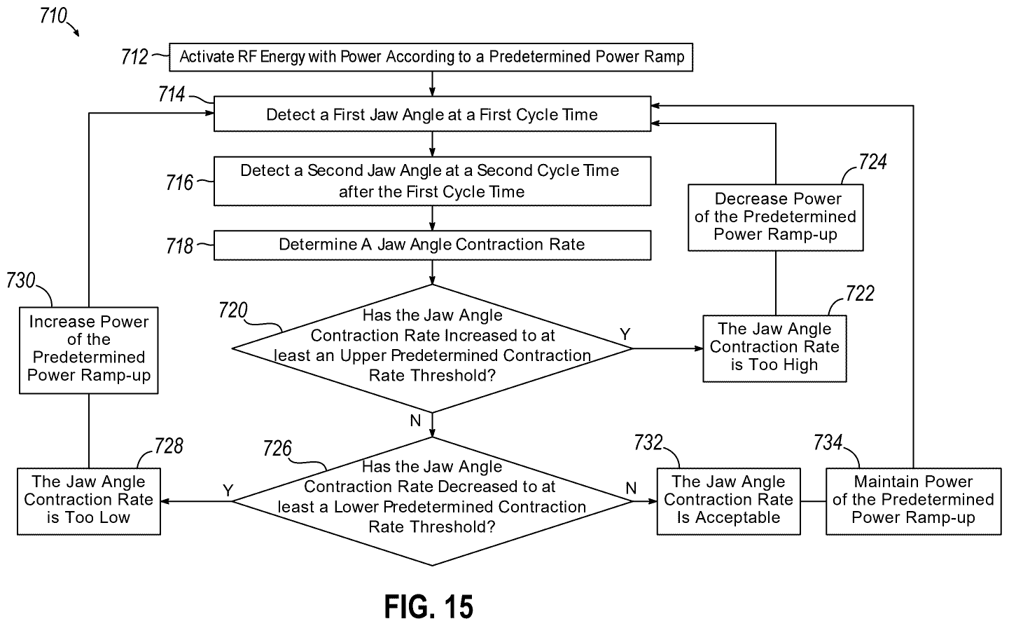

With respect to FIGS. 15-18, an example of a method (710) of adjusting RF energy during the power phase of treatment method (310) includes activating the RF energy with power at a predetermined power ramp-up in a step (712). While various power-ramp ups have been discussed above, jaw angle contraction rate may additionally or alternatively be monitored for selectively increasing or decreasing the applied predetermined power ramp-up as preferably suited to the tissue in real-time thereby increasing the likelihood of achieving the desirable tissue seal. The following method (710) of adjusting RF energy may be incorporated into any method discussed above for achieving selectable predetermined power ramp-ups based at least in part on jaw angle contraction rate.

To this end, following activation of the RF energy in step (712), control unit (102) detects a first jaw angle in a step (714) at a first cycle time with jaw sensor (204) via control unit (102). While RF energy is being applied to the tissue, jaw angle continues to contract as described above, therefore, a second jaw angle is detected in a step (716) at a second cycle time. After the second jaw angle is detected and received by control unit (102), control unit (102) determines a jaw angle contraction rate that occurred from the times that each of the first and second jaws angles were detected in a step (718).

A memory unit of control unit (102) has an upper predetermined contraction rate threshold as well as a lower predetermined contraction rate stored thereon. The upper predetermined contraction rate threshold represents an indication that the jaw angle contraction rate is too high, decreasing the likelihood of achieving the desirable tissue seal, whereas the lower predetermined contraction rate threshold represents an indication that the jaw angle contraction rate is too low, also decreasing the likelihood of achieving the desirable tissue seal. Therefore, control unit (102) compares the determined jaw angle contraction rate of step (718) to the upper predetermined contraction rate threshold and determines whether or not the determined jaw angle contraction rate has increased to at least the upper predetermined contraction rate threshold in a step (720). In the event that the jaw angle contraction rate has increased to at least the upper predetermined contraction rate threshold, then control unit (102) determines that the present jaw angle contraction rate is too high in a step (722). Control unit (102) therefore decrease power of the predetermined power ramp-up in a step (724) and again performs step (714), step (716), step (718), and step (720) for improved tissue scaling.

Alternatively, in the event that the jaw angle contraction rate has not yet reached the upper predetermined contraction rate threshold, control unit (102) then compares the determined jaw angle contraction rate of step (718) to the lower predetermined contraction rate threshold and determines whether or not the determined jaw angle contraction rate has decreased to at least the lower predetermined contraction rate threshold in a step (726). In the event that the jaw angle contraction rate has decreased to at least the predetermined contraction rate threshold, then control unit (102) determines that the present jaw angle contraction rate is too low in a step (728). Control unit (102) therefore increases power of the predetermined power ramp-up in a step (730) and again performs step (714), step (716), step (718), and step (720) and, if applicable, step (726), for improved tissue sealing.

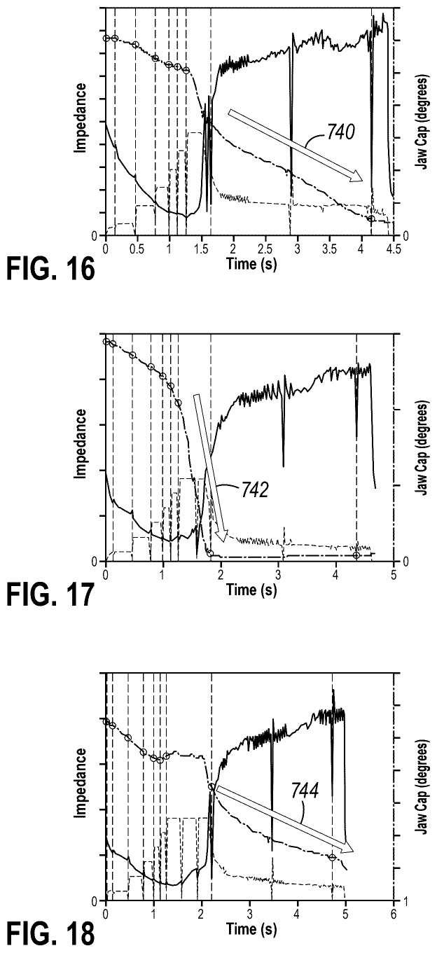

Of course, in the event that the jaw angle contraction rate has not yet decreased to at least the lower predetermined contraction rate threshold, control unit (102) then determines that the jaw angle contraction rate is acceptable in a step (732) such that control unit (102) maintains the present power of the predetermined power ramp-up for the RF energy in a step (734). From step (734), the method returns to step (714) to repeat step (716), step (718), step (720), and step (726) as applicable, thereby continuously correcting the jaw angle contraction rate to retain the jaw angle contraction rate between the upper and lower correction rate thresholds for selectively adjusting the power of the predetermined ramp-ups and increasing the likelihood of achieving the desirable tissue seal. By way of example, FIG. 16 shows one such jaw angle contraction rate (740) controlled as discussed above to retain the jaw angle contraction rate between the upper and lower correction rate thresholds. In contrast, FIG. 17 is one example of jaw angle contraction rate (742) that is decreasing too high, whereas FIG. 18 is one example of jaw angle contraction rate (744) that is decreasing too low. It will be appreciated that alternative outcomes may vary in alternative examples, such as with alternative tissues, such that the invention is not intended to be unnecessarily limited to the particular results shown in FIGS. 16-18.

While the above referenced examples of various thresholds stored on memory unit of control unit (102) may each be respectively accessed and applied to a variety of tissues for treatment, in another example, different predetermined thresholds may also be stored on memory unit of control unit (102) for selection during operation based on feedback from the subtherapeutic RF energy applied while preparing the tissue in the initial phase of step (314) (see FIG. 7). For example, control unit (102) may select one predetermined threshold over another predetermined threshold based on a characteristic of the tissue, such as tissue size and/or tissue type. The invention is therefore not intended to be unnecessarily limited to any one threshold for application to tissue.

### IV. Illustrative Combinations

The following examples relate to various non-exhaustive ways in which the teachings herein may be combined or applied. The following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A surgical instrument, comprising: (a) an end effector, including: (i) a first jaw having a first electrode, and (ii) a second jaw having a second electrode and configured to selectively move relative to the first jaw from an open configuration configured to receive a tissue toward a closed configuration to compress the tissue therebetween, wherein the first and second electrodes are configured to apply a radiofrequency (RF) energy through the tissue with the first and second jaws in the closed configuration; and (b) a controller operatively connected to the first and second jaws and configured to: (i) determine an impedance of the tissue while applying the RF energy to the tissue, (ii) determine a jaw gap contraction rate while applying the RF energy to the tissue, wherein the jaw gap contraction rate is based on a rate of change of a gap between the first jaw and the second jaw, (iii) compare the jaw gap contraction rate to at least one predetermined contraction rate threshold, and (iv) adjust a power of the RF energy while applying the RF energy to the tissue in response to the jaw gap contraction rate reaching the predetermined contraction rate threshold.

### Example 2

The surgical instrument of Example 1, wherein the at least one predetermined contraction rate includes an upper predetermined contraction rate threshold, and wherein the controller is further configured to reduce the power of the RF energy while applying the RF energy to the tissue in response to the jaw gap contraction rate increasing to at least the upper predetermined contraction rate threshold.

### Example 3

The surgical instrument of any one or more of Examples 1 through 2, wherein the at least one predetermined contraction rate includes a lower predetermined contraction rate threshold, and wherein the controller is further configured to reduce the power of the RF energy while applying the RF energy to the tissue in response to the jaw gap contraction rate decreasing to at least the lower predetermined contraction rate threshold.

### Example 4

The surgical instrument of Example 3, wherein the controller is further configured to terminate the power of the RF energy in response to the jaw gap contraction rate decreasing to at least the lower predetermined contraction rate threshold.

### Example 5

The surgical instrument of any one or more of Examples 3 through 4, wherein the controller is further configured to compare the measured impedance to a predetermined impedance threshold.

### Example 6

The surgical instrument of Example 5, wherein the controller is further configured to increase the power of the RF energy while applying the RF energy to the tissue in response to the measured impedance being less than the predetermined impedance threshold.

### Example 7

The surgical instrument of any one or more of Examples 5 through 6, wherein the controller is further configured to activate the power of the RF energy according to a predetermined lower power in response to the measured impedance having increased to at least the predetermined impedance threshold.

### Example 8

The surgical instrument of Example 7, wherein the controller is configured to: (i) measure an elapsed time from activating the power of the power of the RF energy according to the predetermined lower power, and (ii) compare the elapsed time to a predetermined time period.

### Example 9

The surgical instrument of any one or more of Examples 5 through 6, wherein the controller is further configured to activate the power of the RF energy according to one of a series of predetermined lower power ramp-ups in response to the measured impedance having increased to at least the predetermined impedance threshold.

### Example 10

The surgical instrument of any one or more of Examples 1 through 9, wherein the at least one predetermined contraction rate threshold includes an upper predetermined contraction rate threshold and a lower predetermined contraction rate threshold, and wherein the controller is further configured to: (i) activate the power of the RF energy according to a predetermined power ramp-up, (ii) in response to the jaw gap contraction rate increasing to at least the upper predetermined contraction rate threshold, determining that the jaw gap contraction rate is relatively high and therefore decreasing the power of the predetermined power ramp-up, and (iii) in response to the jaw gap contraction rate decreasing to at least the lower predetermined contraction rate threshold, determining that the jaw gap contraction rate is relatively low and therefore increasing the power of the predetermined power ramp-up.

### Example 11

A surgical instrument, comprising: (a) an end effector, including: (i) a first jaw having a first electrode, and (ii) a second jaw having a second electrode and configured to selectively move relative to the first jaw from an open configuration configured to receive a tissue toward a closed configuration to compress the tissue therebetween, wherein the first and second electrodes are configured to apply a radiofrequency (RF) energy through the tissue with the first and second jaws in the closed configuration; and (b) a controller operatively connected to the first and second jaws and configured to: (i) activate a power of the RF energy according to a predetermined power, (ii) detect a first jaw gap between the first and second jaws in the closed configuration with tissue received therebetween and the power of the RF energy activated, (iii) after detecting the first jaw gap, detect a second jaw gap between the first and second jaws in the closed configuration with tissue received therebetween and the power of the RF energy activated, (iv) determine a jaw gap contraction rate based on the first and second jaw gaps, (v) compare the jaw gap contraction rate to at least one predetermined contraction rate threshold, and (iv) adjust the power of the RF energy while applying the RF energy to the tissue in response to the jaw gap contraction rate reaching the predetermined contraction rate threshold.

### Example 12

The surgical instrument of Example 11, wherein the at least one predetermined contraction rate includes an upper predetermined contraction rate threshold, and wherein the controller is further configured to reduce the power of the RF energy while applying the RF energy to the tissue in response to the jaw gap contraction rate increasing to at least the upper predetermined contraction rate threshold.

### Example 13

The surgical instrument of any one or more of Examples 11 through 12, wherein the at least one predetermined contraction rate includes a lower predetermined contraction rate threshold, and wherein the controller is further configured to reduce the power of the RF energy while applying the RF energy to the tissue in response to the jaw gap contraction rate decreasing to at least the lower predetermined contraction rate threshold.

### Example 14

The surgical instrument of any one or more of Examples 11 through 13, wherein the controller is further configured to determine an impedance of the tissue while applying the RF energy to the tissue.

### Example 15

The surgical instrument of Example 14, wherein the controller is further configured to compare the measured impedance to a predetermined impedance threshold.

### Example 16

The surgical instrument of any one or more of Examples 11 through 15, the at least one predetermined contraction rate threshold includes an upper predetermined contraction rate threshold and a lower predetermined contraction rate threshold, and wherein the controller is further configured to: (i) activate the power of the RF energy according to a predetermined power ramp-up, (ii) in response to the jaw gap contraction rate increasing to at least the upper predetermined contraction rate threshold, determining that the jaw gap contraction rate is relatively high and therefore decreasing the power of the predetermined power ramp-up, and (iii) in response to the jaw gap contraction rate decreasing to at least the lower predetermined contraction rate threshold, determining that the jaw gap contraction rate is relatively low and therefore increasing the power of the predetermined power ramp-up.

### Example 17

The surgical instrument of any one or more of Examples 11 through 16, further comprising a shaft assembly projecting proximally from the end effector.

### Example 18

The surgical instrument of any one or more of Examples 11 through 17, further comprising a body projecting proximally from the shaft assembly.

### Example 19

The surgical instrument of any one or more of Examples 11 through 18, wherein the end effector further includes a knife member translatably positioned in at least one of the first and second jaw and configured to sever seal tissue compressed between the first and second jaws in the closed configuration.

### Example 20

A method of adjusting a power of a radiofrequency (RF) energy applied to a tissue via a surgical instrument, the surgical instrument including: (a) an end effector, including: (i) a first jaw having a first electrode, and (ii) a second jaw having a second electrode and configured to selectively move relative to the first jaw from an open configuration configured to receive a tissue toward a closed configuration to compress the tissue therebetween, wherein the first and second electrodes are configured to apply the RF energy through the tissue with the first and second jaws in the closed configuration; and (b) a controller operatively connected to the first and second jaws, the method comprising: (a) determine an impedance of the tissue while applying the RF energy to the tissue; (b) determining a jaw gap contraction rate while applying the RF energy to the tissue, wherein the jaw gap contraction rate is based on a rate of change of a gap between the first jaw and the second jaw; (c) comparing the jaw gap contraction rate to at least one predetermined contraction rate threshold; and (d) adjust a power of the RF energy while applying the RF energy to the tissue in response to the jaw gap contraction rate reaching the predetermined contraction rate threshold.

### V. Miscellaneous

Any one or more of the teaching, expressions, embodiments, examples, etc. described herein may be combined with any one or more of the teachings, expressions, embodiments, examples, etc. described in U.S. Pat. App. No. [Atty. Ref. No. END9573USNP1], entitled “Electrosurgical Instrument with Impedance Spectroscopy and Method of Monitoring State of Instrument Jaws and Tissue,” filed on even date herewith; U.S. Pat. App. No. [Atty. Ref. No. END9574USNP1], entitled “Electrosurgical Instrument with Jaw Status Monitoring and Method of Adjusting Energy Activation,” filed on even date herewith; U.S. Pat. App. No. [Atty. Ref. No. END9576USNP1], entitled “Electrosurgical Instrument and Method of Detecting Tissue Accumulation on End Effector,” filed on even date herewith; U.S. Pat. App. No. [Atty. Ref. No. END9577USNP1], entitled “Electrosurgical Instrument and Method of Applying Energy,” filed on even date herewith; and/or U.S. Pat. App. No. [Atty. Ref. No. END9609USNP1], entitled “Electrosurgical Instrument and Method of Frequency Monitoring for Sealing Tissue,” filed on even date herewith. The disclosure of each of these applications is incorporated by reference herein.

It should be understood that any of the versions of the instruments described herein may include various other features in addition to or in lieu of those described above. By way of example only, any of the devices herein may also include one or more of the various features disclosed in any of the various references that are incorporated by reference herein. Various suitable ways in which such teachings may be combined will be apparent to those of ordinary skill in the art.

While the examples herein are described mainly in the context of electrosurgical instruments, it should be understood that various teachings herein may be readily applied to a variety of other types of devices. By way of example only, the various teachings herein may be readily applied to other types of electrosurgical instruments, tissue graspers, tissue retrieval pouch deploying instruments, surgical staplers, surgical clip appliers, ultrasonic surgical instruments, etc. It should also be understood that the teachings herein may be readily applied to any of the instruments described in any of the references cited herein, such that the teachings herein may be readily combined with the teachings of any of the references cited herein in numerous ways. Other types of instruments into which the teachings herein may be incorporated will be apparent to those of ordinary skill in the art.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions or other disclosure material set forth herein will only be incorporated to the extent that no conflict arises between that incorporated material and the existing disclosure material.

Versions of the devices described above may have application in conventional medical treatments and procedures conducted by a medical professional, as well as application in robotic-assisted medical treatments and procedures. By way of example only, various teachings herein may be readily incorporated into a robotic surgical system such as the DAVINCI™ system by Intuitive Surgical, Inc., of Sunnyvale, California. Similarly, those of ordinary skill in the art will recognize that various teachings herein may be readily combined with various teachings of U.S. Pat. No. 6,783,524, entitled “Robotic Surgical Tool with Ultrasound Cauterizing and Cutting Instrument,” published Aug. 31, 2004, the disclosure of which is incorporated by reference herein, in its entirety.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A surgical instrument, comprising:
(a) an end effector, including:
(i) a first jaw having a first electrode, and
(ii) a second jaw having a second electrode and configured to selectively move relative to the first jaw from an open configuration configured to receive a tissue toward a closed configuration to compress the tissue therebetween, wherein the first and second electrodes are configured to apply a radiofrequency (RF) energy through the tissue with the first and second jaws in the closed configuration; and
(b) a controller operatively connected to the first and second jaws and configured to:
(i) determine an impedance of the tissue while applying the RF energy to the tissue,
(ii) determine a jaw gap contraction rate while applying the RF energy to the tissue, wherein the jaw gap contraction rate is based on a rate of change of a gap between the first jaw and the second jaw,
(iii) compare the jaw gap contraction rate to at least one predetermined contraction rate threshold, and
(iv) adjust a power of the RF energy while applying the RF energy to the tissue in response to the jaw gap contraction rate reaching the predetermined contraction rate threshold.

2. The surgical instrument of claim 1, wherein the at least one predetermined contraction rate includes an upper predetermined contraction rate threshold, and wherein the controller is further configured to reduce the power of the RF energy while applying the RF energy to the tissue in response to the jaw gap contraction rate increasing to at least the upper predetermined contraction rate threshold.

3. The surgical instrument of claim 1, wherein the at least one predetermined contraction rate includes a lower predetermined contraction rate threshold, and wherein the controller is further configured to reduce the power of the RF energy while applying the RF energy to the tissue in response to the jaw gap contraction rate decreasing to at least the lower predetermined contraction rate threshold.

4. The surgical instrument of claim 3, wherein the controller is further configured to terminate the power of the RF energy in response to the jaw gap contraction rate decreasing to at least the lower predetermined contraction rate threshold.

5. The surgical instrument of claim 3, wherein the controller is further configured to compare the measured impedance to a predetermined impedance threshold.

6. The surgical instrument of claim 5, wherein the controller is further configured to increase the power of the RF energy while applying the RF energy to the tissue in response to the measured impedance being less than the predetermined impedance threshold.

7. The surgical instrument of claim 6, wherein the controller is further configured to activate the power of the RF energy according to a predetermined lower power in response to the measured impedance having increased to at least the predetermined impedance threshold.

8. The surgical instrument of claim 7, wherein the controller is configured to:
(i) measure an elapsed time from activating the power of the power of the RF energy according to the predetermined lower power, and
(ii) compare the elapsed time to a predetermined time period.

9. The surgical instrument of claim 6, wherein the controller is further configured to activate the power of the RF energy according to one of a series of predetermined lower power ramp-ups in response to the measured impedance having increased to at least the predetermined impedance threshold.

10. The surgical instrument of claim 1, wherein the at least one predetermined contraction rate threshold includes an upper predetermined contraction rate threshold and a lower predetermined contraction rate threshold, and wherein the controller is further configured to:
(i) activate the power of the RF energy according to a predetermined power ramp-up,
(ii) in response to the jaw gap contraction rate increasing to at least the upper predetermined contraction rate threshold, determining that the jaw gap contraction rate is relatively high and therefore decreasing the power of the predetermined power ramp-up, and
(iii) in response to the jaw gap contraction rate decreasing to at least the lower predetermined contraction rate threshold, determining that the jaw gap contraction rate is relatively low and therefore increasing the power of the predetermined power ramp-up.

11. A surgical instrument, comprising:
(a) an end effector, including:
(i) a first jaw having a first electrode, and
(ii) a second jaw having a second electrode and configured to selectively move relative to the first jaw from an open configuration configured to receive a tissue toward a closed configuration to compress the tissue therebetween, wherein the first and second electrodes are configured to apply a radiofrequency (RF) energy through the tissue with the first and second jaws in the closed configuration; and
(b) a controller operatively connected to the first and second jaws and configured to:
(i) activate a power of the RF energy according to a predetermined power,
(ii) detect a first jaw gap between the first and second jaws in the closed configuration with tissue received therebetween and the power of the RF energy activated,
(iii) after detecting the first jaw gap, detect a second jaw gap between the first and second jaws in the closed configuration with tissue received therebetween and the power of the RF energy activated,
(iv) determine a jaw gap contraction rate based on the first and second jaw gaps,
(v) compare the jaw gap contraction rate to at least one predetermined contraction rate threshold, and
(iv) adjust the power of the RF energy while applying the RF energy to the tissue in response to the jaw gap contraction rate reaching the predetermined contraction rate threshold.

12. The surgical instrument of claim 11, wherein the at least one predetermined contraction rate includes an upper predetermined contraction rate threshold, and wherein the controller is further configured to reduce the power of the RF energy while applying the RF energy to the tissue in response to the jaw gap contraction rate increasing to at least the upper predetermined contraction rate threshold.

13. The surgical instrument of claim 11, wherein the at least one predetermined contraction rate includes a lower predetermined contraction rate threshold, and wherein the controller is further configured to reduce the power of the RF energy while applying the RF energy to the tissue in response to the jaw gap contraction rate decreasing to at least the lower predetermined contraction rate threshold.

14. The surgical instrument of claim 11, wherein the controller is further configured to determine an impedance of the tissue while applying the RF energy to the tissue.

15. The surgical instrument of claim 14, wherein the controller is further configured to compare the measured impedance to a predetermined impedance threshold.

16. The surgical instrument of claim 11, wherein the at least one predetermined contraction rate threshold includes an upper predetermined contraction rate threshold and a lower predetermined contraction rate threshold, and wherein the controller is further configured to:
(i) activate the power of the RF energy according to a predetermined power ramp-up,
(ii) in response to the jaw gap contraction rate increasing to at least the upper predetermined contraction rate threshold, determining that the jaw gap contraction rate is relatively high and therefore decreasing the power of the predetermined power ramp-up, and
(iii) in response to the jaw gap contraction rate decreasing to at least the lower predetermined contraction rate threshold, determining that the jaw gap contraction rate is relatively low and therefore increasing the power of the predetermined power ramp-up.

17. The surgical instrument of claim 11, further comprising a shaft assembly projecting proximally from the end effector.

18. The surgical instrument of claim 17, further comprising a body projecting proximally from the shaft assembly.

19. The surgical instrument of claim 11, wherein the end effector further includes a knife member translatably positioned in at least one of the first and second jaw and configured to sever seal tissue compressed between the first and second jaws in the closed configuration.

20. A method of adjusting a power of a radiofrequency (RF) energy applied to a tissue via a surgical instrument, the surgical instrument including: (a) an end effector, including: (i) a first jaw having a first electrode, and (ii) a second jaw having a second electrode and configured to selectively move relative to the first jaw from an open configuration configured to receive a tissue toward a closed configuration to compress the tissue therebetween, wherein the first and second electrodes are configured to apply the RF energy through the tissue with the first and second jaws in the closed configuration; and (b) a controller operatively connected to the first and second jaws, the method comprising:
(a) determine an impedance of the tissue while applying the RF energy to the tissue;
(b) determining a jaw gap contraction rate while applying the RF energy to the tissue, wherein the jaw gap contraction rate is based on a rate of change of a gap between the first jaw and the second jaw;
(c) comparing the jaw gap contraction rate to at least one predetermined contraction rate threshold; and
(d) adjust a power of the RF energy while applying the RF energy to the tissue in response to the jaw gap contraction rate reaching the predetermined contraction rate threshold.
